(19) United States
(12) Plant Patent Application Publication
(10) Pub. No.: US 2017/0142878 P1
(43) Pub. Date: May 18, 2017

(54) Rosa plant named 'ChewPatout'

(50) ;

(71) Applicant: Christopher Hugh Warner, Brockton (GB)

(72) Inventor: Christopher Hugh Warner, Brockton (GB)

(21) Appl. No.: 14/757,061

(22) Filed: Nov. 12, 2015

(51) Int. Cl. A01H 5/02

(52) U.S. Cl. PLT/108

(57) ABSTRACT

A new and distinct Rosa hybrid named ‘ChewPatout is disclosed, characterized by semi double red to red-purple flowers. Plants are compact, resist blackspot and powdery mildew, and re-bloom easily. The new cultivar is a Rosa typically suited for outdoor ornamental container and landscape use.

Latin name of the genus and species: Rosa hybrid.

Variety denomination: ‘Chewpatout.

### BACKGROUND OF THE INVENTION

The new Rosa cultivar is a product of a planned breeding program conducted by Christopher Hugh Warner in Brockton, Newport Shromshire, United Kingdom. The objective of the propagation was to produce new Rosa varieties for ornamental commercial applications. The cross resulting in this new variety was made during June of 2005.

The seed parent is the unpatented, variety Rosa hybrid ‘Pathfinder’. The pollen parent is Rosa hybrid ‘Radrazz’, U.S. Plant Pat. No. 11,836. The new variety was discovered in June of 2007 by the inventor in a group of seedlings resulting from the 2005 crossing, in a research greenhouse in Brockton, Newport Shromshire, United Kingdom.

Asexual reproduction of the new cultivar was performed by vegetative cuttings. This was first performed at a research greenhouse in Brockton, Newport Shromshire in July 2008 and has shown that the unique features of this cultivar are stable and reproduced true to type in successive generations.

### SUMMARY OF THE INVENTION

The cultivar ‘ChewPatout’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘ChewPatout’ These characteristics in combination distinguish ‘ChewPatout’ as a new and distinct Rosa cultivar:
1. Bright red flowers
2. Good resistance to powdery mildew and blackspot of garden roses.
3. Easy repeat flowering
4. Semi-double flowers, with 15 to 19 petals per flower.

### PARENTAL COMPARISON

The new variety differs from the seed parent in the following:
1. The new variety has red to red-purple colored petals; the seed parent has light pink petals.
2. The new variety has a smaller diameter flower than the seed parent.

The new variety differs from the pollen parent in the following:
1. The new variety has red to red-purple colored petals; the pollen parent has lighter pink petals.
2. The new variety has more petals per flower than the seed parent.
3.

[symbol]

### COMMERCIAL COMPARISON

Plants of the new cultivar ‘ChewPatout’ are similar to the commercial variety Rosa hybrid ‘Meiboulka’ U.S. Plant Pat. No. 19,258, in most horticultural characteristics. ‘However, the new variety differs from ‘Meiboulka’ in the following:
1. Mature upper surface petal color of ‘ChewPatout’ is red-purple 60A, flower color of ‘Meiboulka’ is red-purple 63A, with Red 53A
2. Flowers of the new variety have approximately 15 to 19 petals, flowers of ‘Meiboulka’ average 5 to 7 petals.

### BRIEF DESCRIPTION OF THE PHOTOGRAPHS

The accompanying color photographs illustrate the typical characteristics of the new variety ‘ChewPatout’.

FIG. 1 Illustrates a typical plant of ‘ChewPatout’ grown in the ground, at approximately 2 year old.

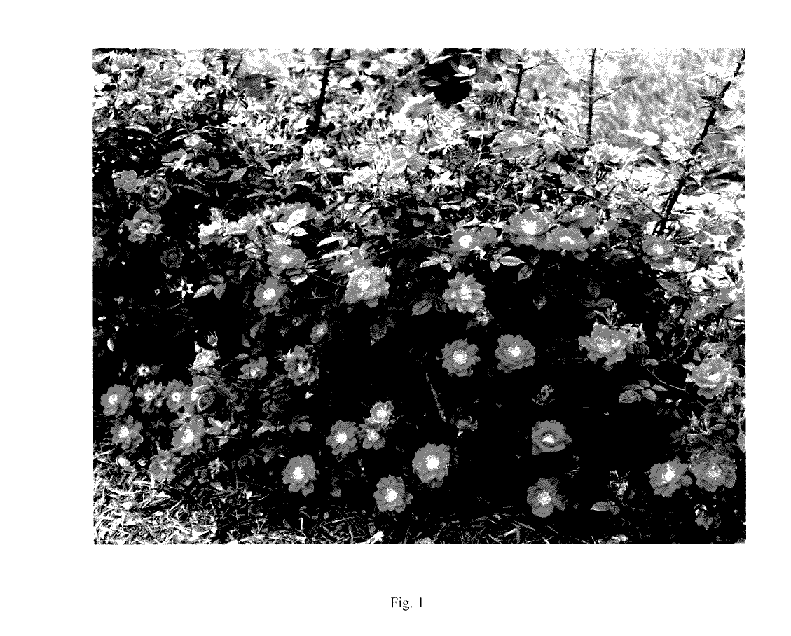

FIG. 2 illustrates a close up view of typical flowers and foliage of the new variety.

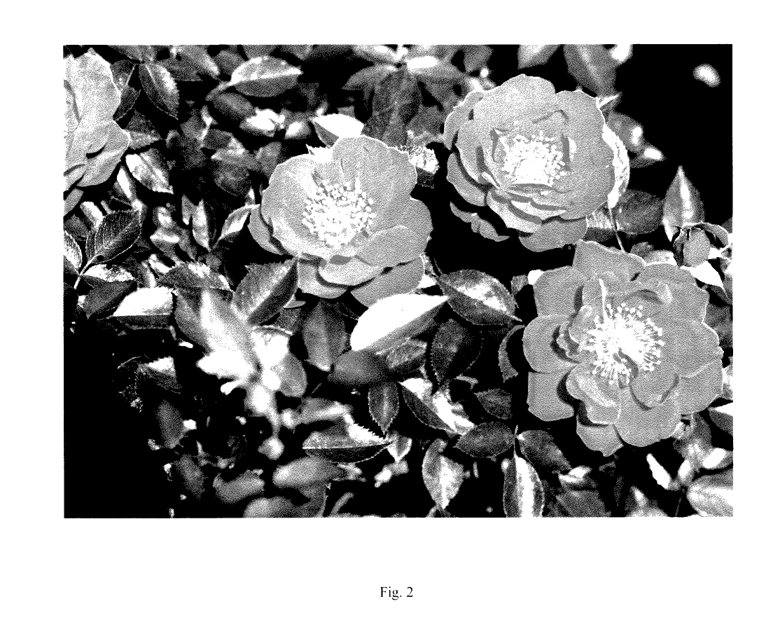

The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe ‘ChewPatout’ plants grown in a poly house greenhouse in Grand Haven, Mich. The growing temperature ranged from 5° C. to 10° C. at night and 18° C. to 27° C. during the day. No artificial light, photoperiodic treatments or chemical treatments were given to the plants. Plants were note pinched and were grown under shade cloth. Measurements and numerical values represent averages of typical flowering types.
Botanical classification: Rosa hybrid ‘ChewPatout’.
Age of the plant described: Approximately 2 to 3 years.
Container size of the plant described: 2 gallon.
Propagation:
   Typical method.—Softwood cuttings.
   Time to initiate rooting.—Approximately 30 days in the summer at about 20° C.
   Time to produce a rooted plantlet.—Approximately 120 days at 20 ° C. or higher.
   Root description.—Moderately dense roots becoming slightly woody with age. Roots colored white when young, brown with age. Not accurately assigned a color with R.H.S. chart.
Plant:
   Plant habit.—Compact, mounding garden rose.
   Height.—58 cm.
   Plant spread.—50 cm.
   Growth rate.—Moderate.
   Branching characteristics.—Densely branched.
   Branching characteristics.—
   Length of primary lateral branches.—48 cm.
   Diameter of lateral branches.—0.6 cm.
   Quantity of lateral branches.—Varies, average 24.
   Branching arrangement.—Basal.
   Thorns.—Number of thorns per linear centimeter: 3-4 Shape: Narrow deltoid, bent downward Base size: Average 0.7 cm Height: Average 0.8 cm Apex: Acicular Color: Immature: Near RHS Yellow-Green 145A on underside and near RHS Greyed-Red 181A on top of thorn Mature: Near RHS Yellow-Green 145A on underside and near RHS Greyed-Red 181A on top of thorn.
Foliage:
   Leaf.—Arrangement: Alternate, compound. Quantity: About 35 per lateral branch. Average Length: About 7.5 cm Average Width: About 4.5 cm.
   Leaflet.—Quantity: 5-7 per leaf Average Length: 2.5 cm Average Width: 2.0 cm Shape of blade: elliptic Apex: Acute to apiculate Base: Rounded Margin: Serrulate Texture of top surface: smooth, glossy. Texture of bottom surface: smooth, glossy. Leaf internode length: 3.5 cm Color: Young foliage upper side: Near RHS Green 139A Young foliage under side: Near RHS Green 138A Mature foliage upper side: Near RHS Green 139A Mature foliage under side: Near RHS Green 138A Venation: Type: pinnate Venation color upper side: Midrib near RHS Yellow-Green 145A, other veins near RHS Green 139A Venation color under side: Midrib near RHS Yellow-Green 145B, other veins near RHS Green 138A Petiole: Length: 2.5 cm Width: 0.3 cm Color: Near RHS Yellow-Green 144B Texture: Smooth Leaflet Petiole: Length: 1 mm Width: 1 mm Color: Near RHS Yellow-Green 145A upper surface, 145B lower surface. Texture: Glabrous with micro thorns Stipule: Quantity: 2 Shape: Linear Apex: Acicular Base: Fused along petiole, base truncate Margin: Ciliate Length: 1.2 to 2.2 cm Width: 2 mm Texture: Glabrous, upper and lower surfaces. Color: Upper surface near RHS Yellow-Green 145A, lower surfaces near Yellow-Green 145B.
Flower:
   Natural flowering season.—Spring through summer.
   Flower arrangement.—Single flowers occurring at branch terminus. 1 to 5 flowers per terminus.
   Peduncle.—Length: 2.5 cm Diameter: 0.2 cm Strength: Strong Angle: Approximately 75° Texture: Glabrous, slightly prickly Color: Yellow-Green 144A.
   Petals.—Petal arrangement: semi-double whorl Size: Length: 2.5-3.0 cm Width: 2.5 cm Shape: Obovate Margin: Entire Apex: Retuse Base: Acute Petal quantity: 15-19 Texture: Glabrous, silky.
   Color.—Petals: When opening: Upper surface: Near RHS Red 45B; 0.45 cm diameter at base is near RHS Yellow 13B Lower surface: Near RHS Red 53C; 0.45 cm diameter at base is near RHS Yellow-Orange 14C Fully opened: Upper surface: Near RHS Red-Purple 60A Lower surface: Near RHS Red-Purple 63A Color Changes when aging: Fades to near RHS Red-Purple 61A.
   Bud.—Shape: Ovate Length: About 1.5 cm Diameter: About 9.5 cm Color: Near RHS Yellow-Green 144A Sepals: Length: 2.2 cm Width: 0.5 cm Shape: acuminate Color: Immature plant: Upper side: Near RHS Yellow-Green 145B; center near RHS Green 141A Under side: Near RHS Green 143A Mature Plant: Upper side: Near RHS Yellow-Green 145B; center near Green 141A Under side: Near RHS Green 143A Texture: smooth/pubescent Fragrance: light and sweet.
Reproductive organs:
   Stamens.—Number: 130 Filament length: About 0.8-1.0 cm Filament color: Near RHS Yellow-Orange 17B.
   Anthers.—Length: 0.1 cm Shape: Club-like Color: Near RHS Yellow-Orange 17A Pollen: Quantity: Moderate Color: Near RHS Yellow-Orange 17A.
   Pistil.—Number: 45 Length: Approximately 0.7 cm Style: Length: About 0.5 cm. Color: Near RHS Red 47B Stigma: Shape: clustered Color: Near RHS Yellow-Orange 18B.
   Receptacle.—Height: 15 mm Diameter: 7 mm Shape: Obovate Texture: Glabrous Color: Near RHS Green 144A.
Other characteristics:
   Seeds and fruits.—No fruit and seed set observed to date.
   Disease/pest resistance.—Excellent disease resistance; resists powdery mildew/black spot pathogens. Neither resistant nor susceptible to typical pests of garden roses.
   Temperature tolerance.—Approximate range −31° C. to 38° C.

What is claimed is:

1. A new and distinct cultivar of Rosa hybrid plant named ‘ChewPatout’ as herein illustrated and described.
